(19) United States
(12) Patent Application Publication
(10) Pub. No.: US 2006/0292216 A1
(43) Pub. Date: Dec. 28, 2006

(54) Enteric delivery of (-)-hydroxycitric acid

(76) Inventors: Dallas L. Clouatre, Santa Monica, CA (US); James M. Dunn, Littleton, CO (US); Caroline Dunn, Littleton, CO (US)

Correspondence Address: FOLEY & LARDNER LLP, 111 HUNTINGTON AVENUE, 26TH FLOOR, BOSTON, MA, 02199-7610, US

(21) Appl. No.: 11/371,270

(22) Filed: Mar. 8, 2006

Related U.S. Application Data
(63) Continuation of application No. PCT/US04/29471, filed on Sep. 10, 2004.
Application No. PCT/US04/29471 is a continuation-in-part of application No. 10/660,805, filed on Sep. 11, 2003, now abandoned.

(51) Int. Cl. A61K 31/19; A61K 9/64; A61K 9/24

(52) U.S. Cl. 424/456; 424/472; 514/574

(57) ABSTRACT

The present invention provides stable encapsulated (−)-hydroxycitric acid (“HCA”) dosage unit forms, uses thereof, as well as and methods of making the same. In particular, HCA and the salts, esters and amides of HCA according to the invention are delivered via enteric vehicles, such as enteric-coated tablets, and also enteric and enteric-coated capsules and soft gelatin capsules (softgels). Enteric-coatings may be applied externally to the HCA-containing dosage unit form or, in the case of capsules and soft gelatin capsules, the enteric compound also may be incorporated into the gelatin shell to yield an HCA-containing dosage unit form of the invention. The HCA-containing compositions are protected against acid degradation, lactonization and undesirable ligand binding in select environments. The invention provides HCA-containing dosage unit forms useful to prevent or reduce the symptoms associated with a disease, disorder or condition such as obesity, weight gain, hunger, hyperlipemia, and postprandial lipemia.

### FIELD OF THE INVENTION

The present invention relates to encapsulated (−)-hydroxycitric acid (hereinafter, “HCA”) dosage unit forms, uses thereof, as well as methods of making the same. Specifically HCA, its salts, esters, and amides, are rendered nonreactive to adds via enteric and enteric-coated capsules, soft gelatin capsules (softgels) and tablets.

### BACKGROUND OF THE INVENTION

(−)Hydroxycitric add (HCA) is a naturally-occurring acid found in the fruit of members of the plant genus Garcinia. Free HCA, calcium, magnesium and potassium salts of HCA (i.e., hydroxycitrates, also referred to as HCA) and poorly characterized mixtures of two or more of these minerals have been sold in the American market. Calcium HCA as well as double-metal HCA compositions containing both calcium HCA and sodium HCA (i.e., calcium/sodium salts) were sold as early as 1993. Most of the commercial preparations of HCA sold to date consist of calcium salts of varying degrees of purity or, more recently, poorly characterized mixtures of calcium HCA and potassium HCA salts.

HCA can affect the metabolic functions of mammals, including humans. HCA, as well as several synthetic derivatives of citric acid, can inhibit the production of fatty acids from carbohydrates, suppress appetite, and inhibit weight gain (Sullivan et al., Am. J. Clin. Nutr. 1977; 30: 767). Numerous other benefits have been attributed to the use of HCA, including, but not limited to, an increase in the metabolism of fat stores for energy and an increase in thermogenesis (the metabolism of energy sources to produce body heat in an otherwise wasteful cycle).

The therapeutic use of HCA salts has been limited, however, by their poor absorption and chemical instability at acidic pH, e.g., inactivation of HCA salts via lactonization upon exposure to the acidic milieu of the mammalian gut. HCA in both its preferred form as potassium HCA salt and in its secondarily preferred form as sodium HCA salt is extremely hygroscopic. As such, HCA in its more biologically active forms can typically only be maintained as a powder under controlled conditions without special processing.

Prior methods to manipulate HCA salts failed to accommodate its instability in acid and hygroscopic nature. Without special precautions, HCA in its free acid form and in its potassium and sodium salt forms will bind to numerous other compounds. The binding of HCA to other compounds can affect its bioavailability to a subject, e.g., the result is HCA less assimilated by a subject.

There remains a need for HCA compositions in dosage forms, e.g., tablets, capsules and soft-gelatin capsules, that avoid rapid degradation and sequestration of HCA administered orally to a subject.

### SUMMARY OF THE INVENTION

The present invention relates to encapsulated HCA-containing compositions and methods of making the same. Specifically HCA, its salts, esters, and amides, are rendered nonreactive to acids via enteric and enteric-coated capsules, soft gelatin capsules (softgels), tablets, and microencapsulation of HCA-containing material prior to punching tablets. The present invention overcomes problems with regard to the use of the potassium, sodium and other salts, esters and amides of HCA. Specifically, the HCA-containing composition of the invention, when orally ingested, is delivered protected against acid degradation, lactonization and undesirable ligand binding such as takes place when HCA is exposed to acidic environments or other challenging conditions.

In one embodiment, the invention provides an enteric HCA-containing dosage unit form comprising HCA and one or more acid-resistant hydrophobic polymer wherein the acid-resistant hydrophobic polymer is present in an enteric coating. In another embodiment, the invention provides an enteric HCA-containing dosage unit form, comprising HCA, one or more acid-resistant hydrophobic polymer; and one or more plasticizer, wherein the acid-resistant hydrophobic polymer and plasticizer are present in an enteric coating. The plasticizer present in the enteric HCA-containing dosage unit form of the invention can be acetylated glycerides; diethylphthalate; triethyl citrate; tributyl citrate; and triacetin. The enteric HCA-containing dosage unit form can contain HCA as HCA free acid; HCA salts; HCA amide; HCA ester, or any combination thereof. In one embodiment, enteric HCA-containing dosage unit form of the invention contains a mixture of potassium HCA and magnesium HCA. In one embodiment, the potassium HCA and magnesium HCA are present in the enteric HCA-containing dosage unit form of the invention in amounts to give a potassium to magnesium cation ratio of about 20 to 1. In one embodiment, the potassium HCA and magnesium HCA are present in the enteric HCA-containing dosage unit form of the invention in amounts to give a potassium to magnesium cation ratio of about 10 to 1. In one embodiment, the potassium HCA and magnesium HCA are present in the enteric HCA-containing dosage unit form of the invention in amounts to give a potassium to magnesium cation ratio of about 5 to 1. In one embodiment, the potassium HCA and magnesium HCA are present in the enteric HCA-containing dosage unit form of the invention in amounts to give a potassium to magnesium cation ratio of about 3 to 1. In one embodiment, the HCA is included in a liquid in the enteric HCA-containing dosage unit form. Such liquids may include, an oil; polyethylene glycol; polyethylene glycol; poloxamers; glycol esters; and acetylated monoglycerides of various molecular weights. The enteric HCA-containing dosage unit form can contain cellulose acetate phthalate; ethyl cellulose; zein; acrylic polymers; diethyl phthalate; acetylated glycerides; hydroxymethylpropylmethyl cellulose phthalate; polyvinyl acetate phthalate; cellulose acetate trimalleate; acrylic polymer plasticizers; polymers of poly lactic acid; polymers of glycolic acid; Eudragit methacrylic acid and methacrylic acid esters; Resomer® RG enteric polymer; shellac, and mixtures thereof. The enteric HCA-containing dosage unit form of the invention can be in the form of a tablet; capsule; and soft-gelatin capsule. In one embodiment, the enteric coating is applied to the enteric HCA-containing dosage unit form of the invention in an amount from about 1% to about 25% of the weight of the drug core of the enteric HCA-containing dosage unit form. In one embodiment, the enteric coating is applied to the enteric HCA-containing dosage unit form of the invention in an amount from about 1% to about 10% of the weight of the drug core of the enteric HCA-containing dosage unit form. In one embodiment, the enteric coating is applied to the enteric HCA-containing dosage unit form of the invention in an amount from about 2% to about 8% of the weight of the drug core of the enteric HCA-containing dosage unit form. In one embodiment, the acid-resistant hydrophobic polymer is present in the shell of an enteric HCA-containing dosage unit form capsule of the invention in an amount from about 1% to about 25% of the weight of the drug core of the enteric HCA-containing dosage unit form capsule. In one embodiment, the acid-resistant hydrophobic polymer is present in the shell of an enteric HCA-containing dosage unit form capsule of the invention in an amount from about 1% to about 10% of the weight of the drug core of the enteric HCA-containing dosage unit form capsule. In one embodiment, the acid-resistant hydrophobic polymer is present in the shell of an enteric HCA-containing dosage unit form capsule of the invention in an amount from about 2% to about 8% of the weight of the drug core of the enteric HCA-containing dosage unit form capsule.

In one embodiment, the enteric (−)-hydroxycitrate-containing dosage unit form contains (−)-hydroxycitrate and one or more cyclodextrins. The one or more cyclodextrins can include, e.g., alpha-cyclodextrin; beta-cyclodextrin; gamma-cyclodextrin; and hydroxy-propyl beta-cyclodextrin, or any combination thereof. In one embodiment, is cyclodextrin is present in an amount from about 0.1% to about 25% of the total weight of the enteric (−)-hydroxycitrate-containing dosage unit form. In another embodiment, the cyclodextrin is present in an amount from about 0.5% to about 10% of the total weight of the enteric (−)-hydroxycitrate-containing dosage unit form. In another embodiment, the cyclodextrin is present in an amount from about 1% to about 8% of the total weight of the enteric (−)-hydroxycitrate-containing dosage unit form.

In one embodiment, the invention provides a pharmaceutical composition comprising an enteric HCA-containing dosage unit form and a pharmaceutically-acceptable carrier.

In one embodiment, the invention provides a method of suppressing the appetite in a subject, the method comprising administering to a subject in which appetite suppression is desired an enteric HCA-containing composition of the invention in an amount sufficient to suppress the appetite in the subject.

In one embodiment, the invention provides a method of reducing the cytoplasmic citrate lyase activity in a subject, the method comprising administering to a subject in which reducing cytoplasmic citrate lyase activity is desired an enteric HCA-containing dosage unit form of the invention in an amount sufficient to reduce the citrate lyase activity.

In one embodiment, the invention provides a method of increasing the fat metabolism in a subject, the method comprising administering to a subject in which increased fat metabolism is desired an enteric HCA-containing dosage unit form of the invention in an amount sufficient to increase fat metabolism.

In one embodiment, the invention provides a method of inducing weight-loss in a subject, the method comprising administering to a subject in which weight-loss is desired an enteric HCA-containing dosage unit form of the invention in an amount sufficient to induce weight-loss.

In one embodiment, the invention provides a method of reducing blood lipids and postprandial lipemia in a subject, the method comprising administering to a subject in which reduced blood lipids and postprandial lipemia is desired an enteric HCA-containing dosage unit form of the invention in an amount sufficient to reduce blood lipids and postprandial lipemia.

### DETAILED DESCRIPTION

I. Definitions

A “subject,” as used herein, is preferably a mammal, such as a human, but can also be an animal, e.g., domestic animals (e.g., dogs, cats and the like), farm animals (e.g., cows, sheep, pigs, horses and the like) and laboratory animals (e.g., rats, mice, guinea pigs and the like).

An “effective amount” of an HCA-containing compound of the invention, as used herein, is a quantity sufficient to achieve a desired therapeutic and/or prophylactic effect, for example, an amount which results in the prevention of or a decrease in the symptoms associated with a disease, disorder or condition that is being treated, e.g., obesity, weight gain, hunger, hyperlipemia, postprandial lipemia. The amount of an HCA-containing composition of the invention administered to the subject will depend on the type and seventy of the disease, disorder or condition, and on the characteristics of the individual, such as general health, age, sex, body weight and tolerance to drugs. It will also depend on the degree, severity and type of disease. The skilled artisan will be able to determine appropriate dosages depending on these and other factors. Typically, an effective amount of the HCA-containing compound of the invention, sufficient for achieving a therapeutic or prophylactic effect, range from about 0.000001 mg per kilogram body weight per day to about 1,000 mg per kilogram body weight per day. Preferably, the dosage ranges are from about 0.0001 mg per kilogram body weight per day to about 100 mg per kilogram body weight per day. The HCA-containing compound of the invention can also be administered alone, or in combination, with one or more additional therapeutic compounds or various encapsulation agents.

It advantageous to formulate oral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of HCA and the particular therapeutic effect to be achieved, and the limitations inherent in the art of compounding such an active compound for the treatment of individuals. The pharmaceutical compositions can be included in a container, pack, or dispenser together with instructions for administration. Typically, an oral dose is taken once to four-times daily, until symptom relief is apparent. The compounds of the present invention can also be administered in combination with each other, or with one or more additional therapeutic compounds. The compounds of the present invention are useful as dietary supplements.

The references cited in this application are incorporated by reference herein in their entireties.

General

The U.S. Pat. No. 6,447,807, granted to Clouatre et al., is directed to methods of coating and encasing HCA compounds in acid-resistant hydrophobic polymers to produce HCA granulate resistant to environmental moisture, lactonization, and undesirable binding. It is an object of the present invention to avoid directly applying enteric-coatings to HCA compounds via blending an acid-resistant hydrophobic polymer to render them resistant to degradation or sequestration in the stomach. These methods are advantageous to avoid excessive contact of acid-resistant polymer with HCA compound in a dosage vehicle, particularly where such contact leads to the over-sequestration of HCA, preventing efficient absorption by, or contact with, the tissue(s) of a subject in need of HCA.

Accordingly, the present invention teaches application, e.g., external application or incorporation of the enteric-coating into the shell of a capsule, of select enteric compounds, e.g., of acid-resistant polymers, to dosage forms of HCA, e.g., tablets, capsules, and soft-gelatin capsules (i.e., HCA-containing dosage unit forms). The application of these select enteric-coatings to dosage forms containing potassium HCA or other salts and mixtures of salts of HCA and HCA derivatives, such as amides and esters, yields a dosage delivery form with a more favorable delivery profile, e.g., tissue site of HCA-delivery to a subject and level of bioavaliable HCA compound absorbed by a subject, relative to the absorption of uncoated HCA compound. That is, the invention provides methods to render non-hygroscopic and stable, e.g., not prone to lactonization, or acid-catalyzed degradation, or sequestration by agents that inhibit their absorption or lead to their excretion, the otherwise hygroscopic chemical forms of HCA. These chemical forms include, but are not limited to, e.g., HCA, its salts and other derivatives. As such, when ingested orally, the HCA contained in the dosage form, e.g., tablets, capsules, and soft gelatin capsules, is resistant to degradation and other undesirable changes in the upper digestive tract, e.g., stomach, and, thus, is presented to the intestinal lumen to provide advantages in absorption.

In one embodiment of the invention, the HCA-containing dosage unit form of the invention is formulated as an enteric-coated tablet containing one or more of HCA salt, ester, amide, or combination thereof. In another embodiment of the invention, the HCA-containing dosage unit form of the invention is formulated as an enteric-coated capsule containing one or more of HCA salt, ester, amide, or combination thereof. In another embodiment of the invention, the HCA-containing dosage unit form of the invention is formulated as a enteric-coated soft-gelatin capsule containing one or more of HCA salt, ester, amide, or combination thereof. The HCA salt of the invention can be a double metal HCA salt, i.e., an HCA salt with more than one type of metal coordinated with the HCA, e.g., calcium/potassium salt in another embodiment of the invention, one or more enteric compounds, e.g., acid-resistant polymer(s), are applied to the exterior surface of the HCA-containing tablet, capsule or soft-gelatin capsule, i.e., softgels. In another embodiment of the invention, one or more enteric compounds, e.g., acid-resistant polymer(s), are incorporated into the gelatin shell. In another embodiment of the invention, one or more enteric compounds, e.g., acid-resistant polymer(s), are both incorporated into the gelatin shell and applied to the exterior surface of an HCA-containing capsule or soft-gelatin capsule. In another embodiment of the invention, one or more enteric compounds, e.g., add-resistant polymer(s), are sequentially applied as layers to the external surface the HCA-containing, tablet, capsule or soft-gelatin capsule.

Characteristics of HCA and HCA Salts

Early work ascribed the weight loss benefit to HCA, its salts and its lactone form. See generally, U.S. Pat. No. 3,764,692 granted to John M. Lowenstein. One commonly offered explanation for the biological and therapeutic effects of HCA is the inhibition of cytoplasmic (cytosolic) ATP-citrate lyase (D. Clouatre and M. E. Rosenbaum, The Diet and Health Benefits of HCA (Hydroxycitric Acid), 1994). In subsequent studies the lactone form of HCA was shown to be far less effective than the sodium salt form of HCA for weight loss purposes. In part because the lactone form lacks the proper affinity for ATP-citrate lyase, known to be a target of the actions of HCA (Lowenstein and Brunengraber, Methods Enzymol. 1981;72:486-97). The sodium salt of HCA is very hygroscopic, however, and is not well-suited to formulation in a stable oral dosage unit form. Under conditions that promote lactonization (e.g., acidic conditions), free HCA undergoes rapid inactivation. Indeed, inclusion of currently available mineral salts of HCA in a prepared beverage of acidic pH leads to the development of HCA lactone over time.

The use of free HCA concentrate in food products has been described in U.S. Pat. No. 5,536,516, but it does not teach any particular advantage for the use of HCA in weight loss or for other medicinal purposes. Even brief exposure of the potassium and sodium salts of HCA to acidic conditions or flavored beverages results in chemical changes in these HCA salts. In some cases the beverages actually change color upon addition of potassium HCA or sodium HCA salts. Calcium and double-metal HCA salts are not immune to these undesirable changes upon exposure to low pH environments.

Free HCA is extremely ionic and does not pass readily through the gut membrane. The free acid form of HCA can be sequestered by binding soluble and insoluble fibers as well as by many other compounds, thus rendering HCA biologically unavailable. There is evidence that the free HCA and HCA lactone are both irritating to the gastrointestinal tissues if consumed regularly in large amounts.

Generally, calcium HCA and magnesium HCA salts, either alone or in the form of various mixtures together, or in combination with the potassium HCA and sodium HCA salts, are not preferred delivery forms for HCA. Calcium HCA and magnesium HCA salts are also not readily absorbed across the gastrointestinal tract because they are poorly soluble in aqueous media. These HCA salts are also reactive with bile acids and fats in the gut and/or are sequestered by binding to soluble and insoluble fibers or other substances in the diet or secreted during digestion (Heymsfield, Steven B, et al. JAMA 1998; 280(18): 1596-1600; Letters, JAMA 1999; 282: 235). For example, the action of stomach acid may free one of the two valences of calcium HCA or magnesium HCA salts for attachment to fats, bile adds, gums, fibers, pectins, and so forth and so on, which is an undesirable outcome. The addition of small amounts of magnesium HCA to potassium HCA, however, improves the transit of potassium HCA across cell membranes. By contrast, calcium, impedes the transit of potassium HCA across cell membranes.

Calcium/potassium HCA (Super CitriMax®) is not well absorbed inasmuch as only 20% of the dose ingested by fasted subjects was detected in the blood using gas chromatography/mass spectroscopy technique (Loe et al., Anal Biochem. 2001, 1;292(i): 148-54). Loe and coworkers reported that the absorption of calcium/potassium HCA (Super CitriMax®) peaked 2 hrs after administration, and that the compound remained in the blood for more than 9 hours after ingestion (Loe et al., FASEB Journal, 15 4:632, Abs. 501.1, 2001). Eating a meal shortly after taking Super CitriMax® reduced its absorption by about 60%. Moreover, animal trials (see U.S. Pat. No. 6,476,071) have further demonstrated that in order for the potassium salt to be maximally effective, the cation must be fully bound to the HCA with only trivial amounts of contaminants, including most other minerals or fibers or sugars.

Calcium HCA salt has some further disadvantages that may limit its therapeutic use. Calcium uptake from the gut is highly regulated and under normal circumstances does not exceed approximately 35% of that found in foods and supplements. The uptake of calcium declines as the dosage of calcium is increased. This may limit the use of calcium HCA where large doses may need to be ingested. For example, for weight loss and other purposes, a minimally effective amount of HCA derived from its calcium salt requires the administration of between 12 g and 15 g of a 50% material. This amount of calcium HCA may lead to undesirably elevated levels of binding and excretion or interference in the uptake of other dietary minerals, such as zinc, aside from presenting difficulties in administration. Double-metal HCA salts in which calcium is one of the cations will share in these disadvantages.

HCA sodium salt has disadvantages for long-term administration to a subject. First, sodium HCA lacks positive metabolic effects with regard to obesity. Second, sodium HCA has potential hypertensive actions. Indeed, several of the early indian-supplied “potassium” salts were, in fact, mixtures of calcium, potassium and sodium (−)-hydroxycitrate. The amount of sodium in these HCA preparations exceeded that allowed in low sodium diets notwithstanding the fact that added sodium is ill-advised in any modern diet. In contrast, potassium HCA does not possess the disadvantages associated with sodium HCA.

A preferred salt of HCA for pharmaceutical use is potassium HCA. The mineral potassium is fully soluble, as is its HCA salt, and is known to possess cell membrane permeability which is 100 times greater than that possessed by sodium. However, the potassium salt of HCA, as is also true of the sodium salt, is extremely hygroscopic and thus not suitable under normal circumstances for the production of dry delivery forms. In drawing moisture to itself, potassium HCA will also tend to bind to available binding sites of compounds in its immediate environment, and this action often later will markedly impede the assimilation of potassium HCA from the gut Potassium HCA is also not suitable for most liquid delivery forms inasmuch as potassium HCA in solution, such as in prepared beverages, will slowly lactonize to an equilibrium which is dependent upon the pH.

Select HCA-Containing Compounds and their Delivery

Several international patent applications and U.S. Patents disclose HCA-containing compounds and its delivery as calcium, magnesium and admixtures of salts. International patent application WO 99/03464, filed 28 Jan. 1999, is directed to HCA-containing compounds with 14 to 26 wt % calcium HCA, and approximately 24 wt % to 40 wt % potassium HCA or approximately 14 to 24 wt % sodium HCA, or a mixture thereof, each calculated as a percentage of the total HCA content of the composition for use in dietary supplements and food products. Studies assessing such a composition showed that its assimilation is exceedingly poor even when taken on an empty stomach (Loe et al., Anal Biochem. May 1, 2001; 292(1): 148-54) and that eating a meal shortly after taking it reduced its absorption by about 60% (Loe et al., Time Course of Hydroxycitrate Clearance in Fasting and Fed Humans, FASEB Journal, 15, 4: 632, Abs. 501.1, 2001). Further, studies comparing the effect of various HCA-containing compounds on body weight and food intake in a rat obesity model showed that a test composition of calcium/potassium HCA salt identical to that described by WO 99/03464 was inferior compared to potassium HCA salt in reducing weight gain in middle-aged rats fed a 30% fat diet (see U.S. Pat. No. 6,476,071 B1). Specifically, at the level of intake used experimentally on a 30% fat diet, potassium HCA increased protein as a percentage of body weight while reducing fat as a percentage of body weight. In contrast, the calcium/potassium salt HCA test composition increased fat and reduced protein as percentages of body weight.

International patent application WO 00/15051 is directed to a method of making calcium HCA more soluble by under-reacting the material, i.e., leaving a substantial amount of HCA lactone in the finished product. This procedure, however, does little to improve the uptake of HCA. The problems with HCA lactone are discussed above, and the HCA lactone in large amounts is known to be irritating (Ishihara et al., J Nutr. December 2000; 130(12): 2990-5). Making calcium soluble, again, does nothing to prevent its reactivity with compounds in the gut, e.g., bile salts, or to improve the general rate of assimilation of calcium HCA. It is noteworthy that the process disclosed in WO 00/15051 was previously disclosed by others in 1997 (Sawada et al., Journal of Japan Oil and Chemicals/Nihon Yukagaku Kaishi December 1997; 46, 12: 1467-1474) and many months earlier in Japanese.

International patent application WO 02/014477 is directed to a composition comprising HCA in combination with either one or both of garcinol and anthocyanin. Garcinol is a common contaminant of HCA products, and thus, it is typically present in the salts which have been used for other clinical studies, i.e., extracts rather than synthesized pure HCA salts. It is unknown whether the additive effect shown in WO 02/014477 extends beyond the mild response reported if higher dosages of either component are ingested. Studies on the effect of Garcinia cambogia-derived flavonoids, however, revealed a dose-dependent, biphasic activity response. (Koshy and Vijayalakshmi Phytother. Res. August 2001; 15(5):395-400). That is, higher doses of the flavonoids were not toxic to test subjects, but they were less effective than lower concentrations of the flavonoids in reducing lipid levels in serum and tissues of test subjects. (Koshy and Vijayalakshmi Phytother. Res. August 2001; 15(5):395-400).

U.S. Pat. No. 6,221,901 is directed to the preparation and uses of magnesium HCA. The high dosage of magnesium HCA required to achieve the indicated results, however, may limit the therapeutic utility of the composition. For example, in order to achieve a hypotensive effect the inventors fed their animals 500 mg/kg magnesium HCA. Using the standard 5:1 multiplier for rat to human data, the dose of magnesium hydroxycitrate employed by Shrivastava et al. is equivalent to a human ingesting 10 mg/kg/day or 7 grams for the average-sized human subject. Of this amount, 45% would be elemental magnesium; hence we have the equivalent of a human ingesting approximately 3.15 grams of magnesium. The Recommended Dietary Allowances, 10th edition (National Research Council, 1989), indicates that most humans begin to suffer diarrhea at more than 350 mg/day. In other words, the test dose used by Shrivastava et al. is nearly 10-times the dose at which side-effects would normally be expected to begin to appear. The induced diarrhea itself would lower blood pressure rapidly.

U.S. Pat. No. 5,783,603 is directed to a technique for the production of potassium HCA. The potassium HCA prepared by this method requires that the milling, sifting, blending and packing of the potassium HCA be carried out in a nitrogen atmosphere as the potassium HCA preparation is otherwise hygroscopic. That is, if left in the open air outside of a humidity-controlled environment, the potassium HCA produced according to that patented method will begin to absorb moisture within a few minutes. This property will limit the use of this material as a component of dry pharmaceutical or nutraceutical preparations.

A fully reacted potassium HCA has a pH greater than 9. Low-pH versions of potassium HCA, i.e., pH of between 7 and 8, are known, however, these forms of potassium HCA are under-reacted, infused with HCA lactone, or suffer similar failings which render them less biologically effective compared with the biological potency of a fully reacted HCA product.

HCA Delivery

The effective delivery of HCA to a subject in need thereof has been limited by the few methods for producing a controlled-release form of HCA, regardless of the salt used. Tests performed to establish the appetite-suppressing effects of HCA demonstrated that a single large oral dose or two divided oral doses totaling one fourth the size of the single dose resulted in a 10% or greater reduction in food consumption in experimental animals fed a high-sugar diet. This result continued over many weeks with the chronic ingestion of HCA. The requirement for at least two divided doses of HCA for efficacy is the only thoroughly established procedure to date.

Giving HCA as multiple doses, as is true of any drug, is inconvenient and is not supported by good patient compliance. Multiple doses given in the form of any of the current salts is also wasteful in that any material delivered to the body which is above the baseline or threshold necessary to produce benefits is simply an excess which is excreted. Controlled release of HCA avoids both excess and waste, on the one hand, and gaps in coverage, on the other hand. Controlled-release makes it possible to simplify the dosage schedule to one daily administration.

As noted above, the potassium salt of HCA is the most efficacious form of HCA to be used for human weight loss and for other pharmaceutical and/or nutraceutical purposes, followed secondarily for these purposes by the sodium salt. In addition, as already indicated, there are benefits to a properly prepared and characterized potassium/magnesium HCA salt in improving transit of HCA across cell membranes.

In one embodiment, the HCA-containing dosage unit form of the invention contains a mixture of potassium HCA and magnesium HCA salts (i.e., potassium/magnesium HCA salt). The potassium to magnesium cation ratio of the HCA salts present in the HCA-containing dosage unit forms of the invention can be varied between a 20:1 and a 3:1 potassium to magnesium ratio. In one embodiment, the potassium/magnesium HCA salt mixture of the HCA-containing dosage unit form has a cation ratio of about 20 to about 1, potassium to magnesium, i.e., a 20:1 potassium: magnesium cation ratio. In one embodiment, the potassium/magnesium HCA salt mixture of the HCA-containing dosage unit form has a cation ratio of about 10 to about 1, potassium to magnesium, i.e., a 10:1 potassium:magnesium cation ratio. In one embodiment, the potassium/magnesium HCA salt mixture of the HCA-containing dosage unit form has a cation ratio of about 5 to about 1, potassium to magnesium, i.e., a 5:1 potassium:magnesium cation ratio. In one embodiment, the potassium/magnesium HCA salt mixture of the HCA-containing dosage unit form has a cation ratio of about 3 to about 1, potassium to magnesium, i.e., a 3:1 potassium:magnesium cation ratio.

The potassium and the sodium salts of HCA present difficulties in handling and manipulation. Potassium HCA is extremely hygroscopic and tends to bind with water in the open air to form a non-palatable paste not suitable for use in tablets, capsules or powders. This material can be admixed with orange juice or water, but requires vacuum pouch sealing under a humidity-controlled atmosphere and is inconvenient for the patient to use. Potassium HCA is reactive with a large number of compounds (tannins, gums, fibers, pectins, and so forth) are thereby readily suffers large losses in pharmacological availability.

Methods of Preparing HCA-Containing Compound of the Invention

By the teachings herein disclosed, HCA acid salts and derivatives can be prepared as capsules, soft gelatin capsules (softgels) and tablets. These forms subsequently can be coated with acid-resistant hydrophobic polymers, which include, but are not limited to, e.g., shellac, cellulose acetate phthalate, hydroxypropyl methylcellulose phthalate, polyvinyl acetate phthalate, cellulose acetate trimaleate, Resomer® RG enteric polymer, Eudragit L55® and other methacrylic acid and methacrylic acid esters, zein and other known enteric products or mixtures thereof, depending upon the properties desired in the finished product. These coatings may also be incorporated directly into shells of hard and soft gelatin capsules. These enteric-coating materials may be applied with or without plasticizers. It is also possible to employ the teachings herein to encapsulate HCA in its free add and lactone forms. (−)Hydroxycitric acid and its lactone, which are liquids, can be made amenable for employment in this invention by first being laid upon a suitable desiccant, e.g., fumed silicon dioxide, as taught by Clouatre et al., U.S. patent application Ser. No. 10/303,117 wherein examples include liquid potassium HCA.

Plasticizers are non-volatile, high boiling liquids used to impart flexibility to otherwise hard or brittle polymeric materials. The addition of a plasticizer in a polymeric film system is generally necessary for the formation of smooth films that are free of cracks and other defects. Plasticizers function by weakening the intermolecular attractions between the polymer chains. These additives have been shown to influence various polymer properties, including the mechanical, adhesive, and drug-release characteristics. Plasticizers useful in the preparation of the enteric coated HCA-containing compositions of the present invention include, but are not limited to, e.g., acetylated glycerides, diethylphthalate, triethyl citrate (TEC), tributyl citrate (TBC), triacetin (GTA or glyceryl triacetate).

Another method is to melt a gelatin mixture with the enteric material in the gelatin solution and make capsules after allowing the melt to fit around forms, which capsules are then filled with HCA and other materials. The HCA powders and granulates may be processed in various manners prior to being placed in the capsules, soft gelatin capsules (softgels) and tablets, for instance, placement in beadlets or microspheres, enteric-coated microspheres, etc. In the case of the soft gelatin capsules, the HCA may be placed first in an oil or other suitable carrier.

In one embodiment of the invention, the percentage of enteric-coating applied to the uncoated HCA-containing dosage form is between about 1% to about 25% of the weight of the drug core of the dosage unit form. In one embodiment of the invention, the percentage of enteric-coating applied to the uncoated HCA-containing dosage form is between about 1% to about 10% of the weight of the drug core of the dosage unit form. In a preferred embodiment of the invention, the percentage of enteric-coating is applied to the uncoated HCA-containing dosage form is between about 2% to about 8% of the weight of the drug core of the dosage unit form.

In one embodiment of the invention, the percentage of enteric-coating incorporated into the shell of an HCA-containing capsule is between about 1% to about 25% of the weight of the drug core of the dosage unit form. In another embodiment of the invention, the percentage of enteric-coating incorporated into the shell of an HCA-containing capsule is between about 1% to about 10% of the weight of the drug core of the dosage unit form. In a preferred embodiment of the invention, the percentage of enteric-coating incorporated into the shell of an HCA-containing capsule is between about 2% to about 8% of the weight of the drug core of the dosage unit form.

The total thickness/weight of the enteric coating is based upon the drug core of the dosage unit form. The drug core of the dosage unit form is the HCA-containing dosage unit form without enteric coating. Work in a low humidity environment is desirable with the potassium HCA and sodium HCA salts.

The present invention employs, unless otherwise indicated, conventional techniques of pharmaceutical formulation, medicinal chemistry, biological testing and the like which are within the reach of one possessing ordinary skill in the art. Such techniques are explained fully in the literature.

It is especially advantageous to formulate the HCA-containing, oral compositions of the invention in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the HCA-containing compound and the particular therapeutic effect to be achieved, and the limitations inherent in the art of compounding such an active compound for the treatment of individuals.

In one embodiment, of the invention the HCA-containing composition is combined with at least one cylodextrin. Cyclodextrins (CDs) are cyclic oligosaccharides commonly composed of six, seven or eight alpha-D-glucose units (α, β, and γ, respectively) which have an overall shape reminiscent of a truncated cone. On account of their relatively hydrophobic interiors, CDs have the ability to form inclusion complexes with a wide range of substrates in aqueous solution. This property of CDs has led to their application in areas as varied as enzyme mimics, catalysis and the encapsulation of drugs (See generally, Chem Rev., 98, issue 5 (1998); Connors, Kans.: The Stability of Cyclodextrin Complexes in Solution. Chem. Rev. 97,1325 (1997); Wenz., G. Angew. Chem. IEE, 33,803 (1994)). Cyclodextrins are useful in the preparation and encapsulation of the compositions of the present invention (see Example 3).

The pharmaceutical compositions can be included in a container, pack, or dispenser together with instructions for administration.

Uses of the HCA-Containing Dosage Unit Forms of the Present Invention

I. Prophylactic and Therapeutic Uses of the HCA-Containing Dosage Unit Forms of the Invention

The HCA-containing dosage unit forms of the present invention are useful in potential prophylactic and therapeutic applications implicated in a variety of disorders, diseases and conditions in a subject including, but not limited to, e.g., obesity, overweight, hunger, deficiencies in fat metabolism, hyperlipemia, and postprandial lipemia. By way of non-limiting example, the compositions of the invention will have efficacy for treatment of subjects suffering from the mentioned disorders mentioned in the Diseases, disorders and conditions, infra.

I. Determination of the Pharmcokinetics or Biological Effect of the HCA-Containing Dosage Unit Forms of the Invention

The pharmacokinetics of HCA-containing dosage unit forms, including absorption, can be determined by measuring the HCA level in the blood of subjects administered an HCA-containing dosage unit form using gas chromatography/mass spectroscopy technique (Loe et al., Anal Biochem. 2001, 1;292(1):148-54) and as further detailed by Loe et al., (FASEB Journal, 2001,15 4:632, Abs. 501.1). The assessment and comparison of the pharmokinetics of test dosage unit forms is well known in the art.

The effect of HCA-containing dosage unit forms on the activity of ATP-citrate lyase can be measured using the ATP-citrate lyase assay procedure as detailed by Houston and Nimmo (Biochim. Biophys. Acta. Feb. 21, 1985; 844(2):233-9). A reduction in ATP-citrate lyase activity in the presence of HCA-containing dosage unit form when compared to the level of ATP-citrate lyase activity observed in the absence of HCA-containing dosage unit form indicates that the HCA-containing dosage unit form inhibits ATP-citrate lyase enzyme.

In various embodiments of the invention, suitable in vitro or in vivo assays are performed to determine the effect of a specific HCA-based therapeutic and whether its administration is indicated for treatment of the affected tissue in a subject.

In various specific embodiments, in vitro assays can be performed with representative cells of the type(s) involved in the patient's disorder, to determine if a given HCA-based therapeutic exerts the desired effect upon the cell type(s). HCA-containing dosage unit forms for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects.

I. Diseases, Disorders and Conditions

The invention provides for both prophylactic and therapeutic methods of treating a subject at risk of (or susceptible to) a disease or having a disorder associated with, e.g., but not limited to, obesity, overweight, deficiencies in lipid metabolism, hyperlipemia, postprandial lipemia, disorders where inhibition of cytoplasmic citrate lyase is advantageous or physical conditions such as hunger.

The HCA-containing dosage unit forms of the present invention are useful to prevent or treat diseases, disorders or conditions where inhibition of ATP-citrate lyase is advantageous, e.g., reduction of cholesterol level. Berkhout et al., (Biochem. J. Nov. 15, 1990; 272(1):181-6) studied the effect of (−)-hydroxycitrate on the activity of the low-density-lipoprotein receptor and 3-hydroxy-3-methylglutaryl-CoA reductase levels in the human hepatoma cell line Hep G2. After 2.5 h and 18 h incubations with HCA at concentrations of 0.5 mM or higher, incorporation of [1,5-14C]citrate into fatty acids and cholesterol was strongly inhibited. It was concluded that this decrease reflected an effective inhibition of ATP citrate-lyase. Cholesterol biosynthesis was decreased to 27% of the control value as measured by incorporations from ³H₂O, indicating a decreased flux of carbon units through the cholesterol-synthetic pathway.

The HCA-containing dosage unit forms of the present invention are useful to prevent or treat diseases or disorders associated with, e.g., but not limited to, obesity; overweight; hyperlipemia; postprandial lipemia; and deficiencies in lipid metabolism, e.g., insulin resistance (Ishihara et al., J Nutr. December 2000; 130(12):2990-5) studied the effect of chronic HCA administration on both carbohydrate utilization and lipid oxidation. The respiratory exchange ratio of test subjects was significantly lower in the HCA group during both resting and exercising conditions. These results suggest that chronic administration of HCA promotes lipid oxidation and spares carbohydrate utilization in test subjects at rest and during running.

Under conditions that elevate de novo lipogenesis in humans, HCA reduced fat synthesis and increased energy expenditure (Kovacs and Westerp-Plantenga, Society for the Study of Ingestive Behavior, Annual Meeting, 2001, Abstr. page 27). The HCA-containing dosage unit forms of the present invention, therefore, are useful in diseases or disorders associated with lipid metabolism.

The HCA-containing dosage unit forms of the present invention are useful to prevent or treat hunger and to promote satiety in a subject as the administration of HCA to subjects has been reported to promote appetite suppression and satiety (Westerterp-Plantenga and Kovacs, Int. J. Obes. Relat Metab. Disord., 2002, 26(6):870-2).

### EXAMPLES

The following examples are intended to be non-limiting illustrations of certain embodiments of the present invention.

### Example 1

Soft gelatin encapsulation was used for oral administration of drugs in liquid form. For this purpose, HCA was provided in a liquid form by suspending it in oils, polyethylene glycol-400, other polyethylene glycols, poloxamers, glycol esters, and acetylated monoglycerides of various molecular weights adjusted such as to insure homogeneity of the capsule contents throughout the batch and to insure-good flow characteristics of the liquid during encapsulation. The soft gelatin shell used to encapsulate the HCA suspension was formulated to impart enteric characteristics to the capsule to ensure that the capsule does not disintegrate until it has reached the small intestine. The basic ingredients of the shell were gelatin, one or more of the enteric materials listed above, plasticizer, and water. Care was exercised in the case of softgels to use the less hygroscopic salts and forms of HCA or to pretreat the more hygroscopic salts to reduce this characteristic. The carrier was adjusted depending on the HCA salt, ester or amide used so as to avoid binding of the ingredients to the carrier.

Plasticizers affect the degree of plasticity, e.g., pliability and flexibility, of enteric-coatings and prevent the shell from becoming too brittle and cracking as the dosage form ages, is exposed to extremely low humidity or is subject to other challenges. In some embodiments of the invention, one or more plasticizers were included in the enteric-coating in an amount(s) sufficient to yield an enteric-coating the will not crack at room temperature for the expected shelf life of the product. Generally, a benchmark for product shelf-life is between about 12 months and about 24 months at the stated label potency and release characterizations.

### Example 2

Many enteric-coatings are used in the pharmaceutical industry. Coatings delivered via organic solvents are preferred when working with the hygroscopic salts of HCA, such as potassium or sodium HCA, although water-based deliveries are acceptable which non-hygroscopic salts, such as calcium HCA. Ammoniated water is also useful as a substitute for organic solvents when non-hygroscopic HCA salts are being employed. Formulations of enteric-coatings useful to make the HCA-containing compounds of the present invention are detailed in Table 1 through Table 4. These coating formulations are useful with all forms of HCA and with hard shell capsules, soft gelatin capsules and properly prepared tablets.

For example, a hard shell capsule was filled with 500 mg potassium-calcium HCA and then coated according to standard procedures using one of these formulations. For hard shell and soft gelatin capsules, the HCA salt, carrier (if needed) and optional additional ingredients were first mixed to prepare the interior formulation. The formulation was then encapsulated and the capsule is coated with a dispersion of enteric-coating components. With tablets, the material was compressed according to procedures well-known in the art The percentage of coating applied was between about 1% and about −10% of the total weight of the capsule or tablet. In a preferred embodiment, the percentage of coating applied was between about 2% and about 10% of the total weight of the capsule or tablet. For unusual conditions of extremely delayed release or the inclusion of certain additional ingredients, the percentage of coating applied can be between about 1% and about 25% of the total weight of the capsule or tablet. Standard techniques for applying enteric-coatings are well-known in the art. Any suitable technique can be used to apply the enteric-coatings to HCA-containing hard shell capsules, soft gelatin capsules and properly prepared tablet.

TABLE 1

| Formulation | % w/w |
|---|---|
| Cellulose acetate phthalate (CAP) | 8.5 |
| Diethyl phthalate | 1.5 |
| Acetone | 45.0 |
| Denatured alcohol | 45.0 |

TABLE 2

| Formulation | % w/w |
|---|---|
| Polyvinyl acetate phthalate | 5.0 |
| Acetylated glycerides | 0.8 |
| Methylene chloride | 47.1 |
| Denatured alcohol | 47.1 |

TABLE 3

| Formulation | % w/w |
|---|---|
| Eudragit methacrylic acid and | 8.0 |
| methacrylic acid esters | |
| Acetone | 46.0 |
| Anhydrous alcohol | 46.0 |
| Plasticizer | as |
| | needed to |
| | prevent |
| | cracking of the |
| | enteric-coating |

TABLE 4

| Formulation | % w/w |
|---|---|
| Hydroxypropyl methylcellulose | 5.0 |
| phthalate | |
| Triacetin | 0.5 |
| Methylene chloride | 47.25 |
| Denatured alcohol | 47.25 |

### Example 3

A number of enzymatically modified starches are available that alter the uptake of organic and other compounds. Cyclodextrins are crystalline water soluble, cyclic, non-reducing, oligosaccharides built up from six, seven, or eight glucopyranose units. Three naturally occurring cyclodextrins are alpha-cyclodextrin, beta-cyclodextrin, and gamma-cyclodextrin. Among these, beta-cyclodextrin is mostly common used. Hydroxy-propyl beta-cyclodextrin is another form commonly employed. They contain a relatively hydrophobic central cavity and hydrophilic outer surface.

Molecules of poorly soluble drugs, rapidly deteriorating flavor substances, volatile fragrances, and so forth can be encapsulated and then released by cyclodextrin molecular encapsulation. Cyclodextrins also prevent drug-drug or drug-additive interactions. The cyclodextrin acts as a chemical basket to entrap the compound and hold it in suspension. In the case of highly ionic substances, such as HCA, the cavity of the cyclodextrin structure holds its payload until it reaches the appropriate release point in the gut. It is possible to use cyclodextrins with an enteric coated granulate of HCA (U.S. Pat. No. 6,447,807). However, it may be less expensive and more convenient to coat the HCA directly with cyclodextrins and then, if desired, to place the cyclodextrin-coated HCA granulate into enteric capsules, or to form tablets that subsequently are enterically coated. In this example, a fluid bed dryer is used to apply −3% beta-cyvlodextrin to HCA powder as summarized in Table 5 and as detailed below.

TABLE 5

| Ingredient | Amount |
|---|---|
| HCA potassium/magnesium | 3.000 kg |
| salt (67.5% HCA content) | |
| beta-Cyclodextrins | 0.090 kg |
| Water for solution | 0.183 kg |
| Total Solids | 3.090 kg |

The beta-cyclodextrin is dissolved in water and used to coat the HCA in fluid bed dryer at a spray rate of 10-12%; outlet temperature of 36.3° C.; inlet temperature of 61.6° C.; auto air pressure of 55 psi; flap of 20%; dry to 45° C. [Outlet Temperature]. Larger batches may require adjustment. Once the HCA has been coated, it is suitable for filling enteric capsules, tableting with excepients as needed and then enterically coated, etc.

### Example 4

### Testing the HCA-Containing Compounds in a Rat Model

An OM rat model is useful to test the biological properties of the HCA-containing dosage unit forms of the invention. Briefly, male OM rats aged 10 weeks are fed a diet in which 30% of the calories are obtained from fat under standard conditions. Groups of 5-10 rats are intubated twice daily with HCA-containing dosage unit forms (e.g., 0.01 mmoles/kg body weight to 1 mole/kg body weight equivalent) or placebo for 60 days. Blood is withdrawn from the tail vein one or more times daily. The pharmacokinetics of HCA-containing dosage unit form, including absorption, is determined by measuring the HCA level in the blood of subjects administered the HCA-containing dosage unit form using gas chromatography/mass spectroscopy technique (Loe et al., Anal Biochem. 2001, 1; 292(1): 148-54) and as further detailed by Loe et al., (FASEB Journal, 2001,154:632, Abs. 501.1). Body weight of the test subjects as well as, blood levels of lipids, hormones and metabolic regulators are measured, e.g., but not limited to, LDL and HDL, glucocorticoids, leptin, insulin, and corticosterone level (see generally, U.S. Pat. No. 6,482,858, issued No. 19, 2002). At the end of the 60 day experimental period, the animals are sacrificed. Experimental parameters such as body weight of the test subjects as well as, blood levels of lipids, hormones and metabolic regulators are measured, e.g., but not limited to, LDL and HDL, glucocorticoids, leptin, insulin, and corticosterone level in test subjects receiving HCA-containing dosage unit form is compared with these experimental parameters in subjects receiving placebo by statistical analysis using the Students t-test (one- or two-tailed P-values) or ANOVA. A P-value of less than or equal to about 0.05 is considered statistically significant. A statistically significant alteration, e.g., increase or decrease, in an experimental parameter of test subjects receiving HCA-containing dosage unit form compared to subjects receiving placebo indicates that the HCA-containing dosage unit form is a form capable of the prevention or treatment of diseases or conditions characterized by alterations in such parameters.

### EQUIVALENTS

From the foregoing detailed description of the invention, it should be apparent that unique HCA-containing dosage unit forms and methods of the same have been described resulting in improved HCA-containing formulations suitable for therapeutic use. Although particular embodiments of the invention have been disclosed herein in detail, this has been done by way of example for purposes of illustration only, and is not intended to be limiting with respect to the scope of the appended claims which follow. In particular, it is contemplated by the inventor that substitutions, alterations, and modifications may be made to the invention without departing from the spirit and scope of the invention as defined by the claims. For instance, the choice of HCA salt, encapsulating agent or the choice of appropriate patient therapy based on these is believed to be matter of routine for a person of ordinary skill in the art with knowledge of the embodiments of the invention described herein.

We claim:

1. An enteric (−)-hydroxycitrate-containing dosage unit form, comprising:
(a) (−)-hydroxycitrate; and
(b) one or more acid-resistant hydrophobic polymer;
wherein the acid-resistant hydrophobic polymer is present in an enteric coating.

2. The enteric (−)-hydroxycitrate-containing dosage unit form of claim 1, wherein the (−)-hydroxycitrate is selected from the group consisting of: (−)-hydroxycitrate free add; (−)-hydroxycitrate salts; (−)-hydroxycitrate amide; (−)-hydroxycitrate ester, or any combination thereof:

3. The enteric (−)-hydroxycitrate-containing dosage unit form according to claim 2, wherein the (−)-hydroxycitrate salts are a mixture of potassium (−)-hydroxycitrate and magnesium (−)-hydroxycitrate.

4. The enteric (−)-hydroxycitrate-containing dosage unit form according to claim 3, wherein the (−)-potassium (−)-hydroxycitrate and magnesium (−)-hydroxycitrate have a potassium to magnesium cation ratio of about 20 to 1.

5. The enteric (−)-hydroxycitrate-containing dosage unit form according to claim 3, wherein the (−)-potassium (−)-hydroxycitrate and magnesium (−)-hydroxycitrate have a potassium to magnesium cation ratio of about 10 to 1.

6. The enteric (−)-hydroxycitrate-containing dosage unit form according to claim 3, wherein the (−)-potassium (−)-hydroxycitrate and magnesium (−)-hydroxycitrate have a potassium to magnesium cation ratio of about 5 to 1.

7. The enteric (−)-hydroxycitrate-containing dosage unit form according to claim 3, wherein the (−)-potassium (−)-hydroxycitrate and magnesium (−)-hydroxycitrate have a potassium to magnesium cation ratio of about 3 to 1.

8. The enteric (−)-hydroxycitrate-containing dosage unit form of claim 1, wherein the (−)-hydroxycitrate is included in a liquid.

9. The enteric (−)-hydroxycitrate-containing dosage unit form of claim 8, wherein the in the liquid is selected from the group consisting of an oil; polyethylene glycol; polyethylene glycol; poloxamers; glycol esters; and acetylated monoglycerides of various molecular weights.

10. The enteric (−)-hydroxycitrate-containing dosage unit form of claim 1, wherein the acid-resistant hydrophobic polymer is selected from the group consisting of cellulose acetate phthalate; ethyl cellulose; zein; acrylic polymers; diethyl phthalate; acetylated glycerides; hydroxymethylpropylmethyl cellulose phthalate; polyvinyl acetate phthalate; cellulose acetate trimalleate; acrylic polymer plasticizers; polymers of poly lactic acid; polymers of glycolic acid; Eudragit methacrylic acid and methacrylic acid esters; Resomer® RG enteric polymer; shellac, and mixtures thereof.

11. The enteric (−)-hydroxycitrate-containing dosage unit form of claim 1, wherein the enteric (−)-hydroxycitrate-containing dosage unit form is in a form selected from the group consisting of a tablet; capsule; and soft-gelatin capsule.

12. The enteric (−)-hydroxycitrate-containing dosage unit form of claim 11, wherein the enteric coating is applied in an amount from about 1% to about 25% of the weight of the drug core of the enteric (−)-hydroxycitrate-containing dosage unit form.

13. The enteric (−)-hydroxycitrate-containing dosage unit form of claim 11, wherein the enteric coating is applied in an amount from about 1% to about 10% of the weight of the drug core of the enteric (−)-hydroxycitrate-containing dosage unit form.

14. The enteric (−)-hydroxycitrate-containing dosage unit form of claim 11, wherein the enteric coating is applied in an amount from about 2% to about 8% of the weight of the drug core of the enteric (−)-hydroxycitrate-containing dosage unit form.

15. The enteric (−)-hydroxycitrate-containing dosage unit form of claim 11, wherein the acid-resistant hydrophobic polymer is present in the shell of a capsule in an amount from about 1% to about 25% of the weight of the drug core of the enterlc (−)-hydroxycitrate-containing dosage unit form.

16. The enteric (−)-hydroxycitrate-containing dosage unit form of claim 11, wherein the acid-resistant hydrophobic polymer is present in the shell of a capsule in an amount from about 1% to about 10% of the weight of the drug core of the enteric (−)-hydroxycitrate-containing dosage unit form.

17. The enteric (−)-hydroxycitrate-containing dosage unit form of claim 11, wherein the acid-resistant hydrophobic polymer is present in the shell of a capsule in an amount from about 2% to about 8% of the weight of the drug core of the enteric (−)-hydroxycitrate-containing dosage unit form.

18. An enteric (−)-hydroxycitrate-containing dosage unit form, comprising:
(a) (−)-hydroxycitrate;
(b) one or more acid-resistant hydrophobic polymer; and
(c) one or more plasticizer,
wherein the add-resistant hydrophobic polymer and plasticizer are present in an enteric coating.

19. The enteric (−)-hydroxycitrate-containing dosage unit form of claim 18, wherein the plasticizer is selected from the group consisting of: acetylated glycerides; diethylphthalate; triethyl citrate; tributyl citrate; and triacetin.

20. The enteric (−)-hydroxycitrate-containing dosage unit form of claim 18, wherein the (−)-hydroxycitrate is selected from the group consisting of: (−)-hydroxycitrate free acid; (−)-hydroxycitrate salts; (−)-hydroxycitrate amide; (−)-hydroxycitrate ester, or any combination thereof.

21. The enteric (−)-hydroxycitrate-containing dosage unit form according to claim 20, wherein the (−)-hydroxycitrate salts are a mixture of potassium (−)-hydroxycitrate and magnesium (−)-hydroxycitrate.

22. The enteric (−)-hydroxycitrate-containing dosage unit form according to claim 21, wherein the (−)-potassium (−)-hydroxycitrate and magnesium (−)-hydroxycitrate have a potassium to magnesium cation ratio of about 20 to 1.

23. The enteric (−)-hydroxycitrate-containing dosage unit form according to claim 21, wherein the (−)-potassium (−)-hydroxycitrate and magnesium (−)-hydroxycitrate have a potassium to magnesium cation ratio of about 10 to 1.

24. The enteric (−)-hydroxycitrate-containing dosage unit form according to claim 21, wherein the (−)-potassium (−)-hydroxycitrate and magnesium (−)-hydroxycitrate have a potassium to magnesium cation ratio of about 5 to 1.

25. The enteric (−)-hydroxycitrate-containing dosage unit form according to claim 21, wherein the (−)-potassium (−)-hydroxycitrate and magnesium (−)-hydroxycitrate have a potassium to magnesium cation ratio of about 3 to 1.

26. The enteric (−)-hydroxycitrate-containing dosage unit form of claim 18, wherein the (−)-hydroxycitrate is in a liquid form.

27. The enteric (−)-hydroxycitrate-containing dosage unit form of claim 18, wherein the in the liquid form of the (−)-hydroxycitrate includes a liquefying agent selected from the group consisting of: an oil; polyethylene glycol; polyethylene glycol; poloxamers; glycol esters; and acetylated monoglycerides of various molecular weights.

28. The enteric (−)-hydroxycitrate-containing dosage unit form of claim 18, wherein the acid-resistant hydrophobic polymer is selected from the group consisting of, cellulose acetate phthalate; ethyl cellulose; zein; acrylic polymers; diethyl phthalate; acetylated glycerides; hydroxymethylpropylmethyl cellulose phthalate; polyvinyl acetate phthalate; cellulose acetate trimalleate; acrylic polymer plasticizers; polymers of poly lactic acid; polymers of glycolic acid; Eudragit methacrylic acid and methacrylic acid esters; Resomer® RG enteric polymer; shellac, and mixtures thereof.

29. The enteric (−)-hydroxycitrate-containing dosage unit form of claim 18, wherein the enteric (−)-hydroxycitrate-containing dosage unit form is in a form selected from the group consisting of: a tablet; capsule; and soft-gelatin capsule.

30. The enteric (−)-hydroxycitrate-containing dosage unit form of claim 29, wherein the enteric coating is applied in an amount from about 1% to about 25% of the weight of the drug core of the enteric (−)-hydroxycitrate-containing dosage unit form.

31. The enteric (−)-hydroxycitrate-containing dosage unit form of claim 29, wherein the enteric coating is applied in an amount from about 1% to about 10% of the weight of the drug core of the enteric (−)-hydroxycitrate-containing dosage unit form.

32. The enteric (−)-hydroxycitrate-containing dosage unit form of claim 29, wherein the enteric coating is applied in an amount from about 2% to about 8% of the weight of the drug core of the enteric (−)-hydroxycitrate-containing dosage unit form.

33. The enteric (−)-hydroxycitrate-containing dosage unit form of claim 29, wherein the acid-resistant hydrophobic polymer is present in the shell of a capsule in an amount from about 1% to about 25% of the weight of the drug core of the enteric (−)-hydroxycitrate-containing dosage unit form.

34. The enteric (−)-hydroxycitrate-containing dosage unit form of claim 29, wherein the acid-resistant hydrophobic polymer is present in the shell of a capsule in an amount from about 1% to about 10% of the weight of the drug core of the enteric (−)-hydroxycitrate-containing dosage unit form.

35. The enteric (−)-hydroxycitrate-containing dosage unit form of claim 29, wherein the acid-resistant hydrophobic polymer is present in the shell of a capsule in an amount from about 2% to about 8% of the weight of the drug core of the enteric (−)-hydroxycitrate-containing dosage unit form.

36. An enteric (−)-hydroxycitrate-containing dosage unit form, comprising (−)-hydroxycitrate and one or more cyclodextrins.

37. The enteric (−)-hydroxycitrate-containing dosage unit form of claim 36, wherein the one or more cyclodextrins is selected from the group consisting of: alpha-cyclodextrin; beta-cylodextrin; gamma-cyclodextrin; and hydroxy-propyl beta-cylodextrin; or any combination thereof.

38. The enteric (−)-hydroxycitrate-containing dosage unit form of claim 36, wherein the (−)-hydroxycitrate is selected from the group consisting of: (−)-hydroxycitrate free acid; (−)-hydroxycitrate salts; (−)-hydroxycitrate amide; (−)-hydroxycitrate ester, or any combination thereof.

39. The enteric (−)-hydroxycitrate-containing dosage unit form according to claim 38, wherein the (−)-hydroxycitrate salts are a mixture of potassium (−)-hydroxycitrate and magnesium (−)-hydroxycitrate.

40. The enteric (−)-hydroxycitrate-containing dosage unit form according to claim 39, wherein the (−)-potassium (−)-hydroxycitrate and magnesium (−)-hydroxycitrate have a potassium to magnesium cation ratio of about 20 to 1.

41. The enteric (−)-hydroxycitrate-containing dosage unit form according to claim 39, wherein the (−)-potassium (−)-hydroxycitrate and magnesium (−)-hydroxycitrate have a potassium to magnesium cation ratio of about 10 to 1.

42. The enteric (−)-hydroxycitrate-containing dosage unit form according to claim 39, wherein the (−)-potassium (−)-hydroxycitrate and magnesium (−)-hydroxycitrate have a potassium to magnesium cation ratio of about 5 to 1.

43. The enteric (−)-hydroxycitrate-containing dosage unit form according to claim 39, wherein the (−)-potassium (−)-hydroxycitrate and magnesium (−)-hydroxycitrate have a potassium to magnesium cation ratio of about 3 to 1.

44. The enteric (−)-hydroxycitrate-containing dosage unit form of claim 36, wherein the enteric (−)-hydroxycitrate-containing dosage unit form is in a form selected from the group consisting of: a tablet; capsule; and soft-gelatin capsule.

45. The enteric (−)-hydroxycitrate-containing dosage unit form of claim 36, wherein the cyclodextrin is present in an amount from about 0.1% to about 25% of the total weight of the enteric (−)-hydroxycitrate-containing dosage unit form.

46. The enteric (−)-hydroxycitrate-containing dosage unit form of claim 36, wherein the cyclodextrin is present in an amount from about 0.5% to about 10% of the total weight of the enteric (−)-hydroxycitrate-containing dosage unit fonm.

47. The enteric (−)-hydroxycitrate-containing dosage unit form of claim 36, wherein the cyclodextrin is present in amount from about 1% to about 8% of the total weight of the enteric (−)-hydroxycitrate-containing dosage unit form.

48. A pharmaceutical composition comprising enteric (−)-hydroxycitrate-containing dosage unit form of claim 1 and a pharmaceutically-acceptable carrier.

49. A pharmaceutical composition comprising enteric (−)-hydroxycitrate-containing dosage unit form of claim 18 and a pharmaceutically-acceptable carrier.

50. A pharmaceutical composition comprising enteric (−)-hydroxycitrate-containing dosage unit form of claim 36 and a pharmaceutically-acceptable carrier.

51. A method of suppressing the appetite in a subject, the method comprising administering to a subject in which appetite suppression is desired the enteric HCA-containing dosage unit form of claim 1 in an amount sufficient to suppress the appetite in the subject.

52. A method of suppressing the appetite in a subject, the method comprising administering to a subject in which appetite suppression is desired the enteric (−)-hydroxycitrate-containing dosage unit form of claim 18 in an amount sufficient to suppress the appetite in the subject.

53. A method of suppressing the appetite in a subject, the method comprising administering to a subject in which appetite suppression is desired the enteric (−)-hydroxycitrate-containing dosage unit form of claim 36 in an amount sufficient to suppress the appetite in the subject.

54. A method of reducing the cytoplasmic citrate lyase activity in a subject, the method comprising administering to a subject in which reducing cytoplasmic citrate lyase activity is desired the enteric (−)-hydroxycitrate-containing dosage unit form of claim 1 in an amount sufficient to reduce the citrate lyase activity.

55. A method of reducing the cytoplasmic citrate lyase activity in a subject, the method comprising administering to a subject in which reducing cytoplasmic citrate lyase activity is desired the enteric (−)-hydroxycitrate-containing dosage unit form of claim 18 in an amount sufficient to reduce the citrate lyase activity.

56. A method of reducing the cytoplasmic citrate lyase activity in a subject, the method comprising administering to a subject in which reducing cytoplasmic citrate lyase activity is desired the enteric (−)-hydroxycitrate-containing dosage unit form of claim 36 in an amount sufficient to reduce the citrate lyase activity.

57. A method of increasing the fat metabolism in a subject, the method comprising administering to a subject in which increased fat metabolism is desired the enteric (−)-hydroxycitrate-containing dosage unit form of claim 1 in an amount sufficient to increase fat metabolism.

58. A method of increasing the fat metabolism in a subject, the method comprising administering to a subject in which increased fat metabolism is desired the enteric (−)-hydroxycitrate-containing dosage unit form of claim 18 in an amount sufficient to increase fat metabolism.

59. A method of increasing the fat metabolism in a subject, the method comprising administering to a subject in which increased fat metabolism is desired the enteric (−)-hydroxycitrate-containing dosage unit form of claim 36 in an amount sufficient to increase fat metabolism.

60. A method of inducing weight-loss in a subject, the method comprising administering to a subject in which weight-loss is desired the enteric (−)-hydroxycitrate-containing dosage unit form of claim 1 in an amount sufficient to induce weight-loss.

61. A method of inducing weight-loss in a subject, the method comprising administering to a subject in which weight-loss is desired the enteric (−)-hydroxycitrate-containing dosage unit form of claim 18 in an amount sufficient to induce weight-loss.

62. A method of inducing weight-loss in a subject, the method comprising administering to a subject in which weight-loss is desired the enteric (−)-hydroxycitrate-containing dosage unit form of claim 36 in an amount sufficient to induce weight-loss.

63. A method of reducing blood lipids and postprandial lipemia in a subject, the method comprising administering to a subject in which reduced blood lipids and postprandial lipemia is desired the enteric (−)-hydroxycitrate-containing dosage unit form of claim 1 in an amount sufficient to reduce blood lipids and postprandial lipemia.

64. A method of reducing blood lipids and postprandial lipemia in a subject, the method comprising administering to a subject in which reduced blood lipids and postprandial lipemia is desired the enteric (−)-hydroxycitrate-containing dosage unit form of claim 18 in an amount sufficient to reduce blood lipids and postprandial lipemia.

65. A method of reducing blood lipids and postprandial lipemia in a subject, the method comprising administering to a subject in which reduced blood lipids and postprandial lipemia is desired the enteric (−)-hydroxycitrate-containing dosage unit form of claim 36 in an amount sufficient to reduce blood lipids and postprandial lipemia.
